(19) United States
(12) Patent Application Publication
(10) Pub. No.: US 2025/0312508 A1
(43) Pub. Date: Oct. 9, 2025

(54) MONITORING METHOD AND SYSTEM FOR MONITORING STERILIZATION EFFECT OF NON-INCINERATION MEDICAL WASTE TREATMENT

(71) Applicant: ZHEJIANG WEIDUN ENVIRONMENTAL TECHNOLOGY CO., LTD., Hangzhou (CN)

(72) Inventors: Weixing CHEN, Hangzhou (CN); Yiming CHEN, Hangzhou (CN); Guanzhong HUANG, Hangzhou (CN)

(73) Assignee: ZHEJIANG WEIDUN ENVIRONMENTAL TECHNOLOGY CO., LTD., Hangzhou (CN)

(21) Appl. No.: 19/171,868

(22) Filed: Apr. 7, 2025

(30) Foreign Application Priority Data
Apr. 7, 2024 (CN) 202410406929.8

(51) Int. Cl. A61L 11/00; C12Q 1/22

(52) U.S. Cl. CPC A61L 11/00; C12Q 1/22

(57) ABSTRACT

Disclosed are a monitoring method and system for monitoring sterilization effect of non-incineration-based medical waste treatment. The method includes: acquiring real-time temperature, target sterilization logarithmic value, and image data within a medical waste treatment chamber; determining whether a sterilization target can be achieved within a preset time based on analysis of these data; maintaining the current sterilization program if the sterilization target is achievable, or adjusting parameters thereof if not. This application enables real-time monitoring and analysis of sterilization efficacy, dynamically determining whether to maintain or adjust the sterilization program, thereby ensuring timeliness, reducing reliance on personnel, and enhancing safety through automated processes.

### CROSS-REFERENCE TO RELATED APPLICATIONS

The present application claims priority to Chinese Patent Application No. 202410406929.8, filed on Apr. 7, 2024, the content of which is incorporated herein by reference in its entirety.

### TECHNICAL FIELD

The present application relates to the technical field of medical waste treatment, and specifically to a monitoring method and system for monitoring sterilization effect of non-incineration medical waste treatment.

### BACKGROUND

In the medical field, a large amount of medical waste is generated daily. Improper handling of such waste may lead to disease transmission, posing serious threats to the environment and human health. Traditional medical waste treatment methods often involve incineration. However, due to potential secondary pollution such as harmful gas emissions and ash residue disposal challenges, non-incineration treatment technologies have gained increasing attention.

Among non-incineration treatment methods, high-temperature steam treatment and microwave disinfection are two mainstream techniques. High-temperature steam treatment utilizes high-temperature, high-pressure steam to sterilize medical waste by destroying microbial cell structures. Microwave disinfection employs thermal and non-thermal effects of microwaves to denature microbial proteins and nucleic acids, thereby achieving microbial inactivation.

In addition to high-temperature steam and microwave disinfection, non-incineration treatment may also utilize frictional heat. Specifically, blades on a rotor controlled by a frequency converter in a sterilization chamber grind solid medical waste into fine particles while generating frictional heat to uniformly heat the waste, ensuring complete sterilization within the chamber.

However, these non-incineration technologies face practical challenges. Specifically, none can monitor sterilization effects in real time. Traditional monitoring relies on manual observation through equipment viewports, which is inefficient and poses significant safety risks. Medical waste may contain pathogenic microorganisms and toxic substances; operational errors or equipment failures during manual observation could lead to hazardous material leakage, endangering personnel and the environment.

Furthermore, manual observation suffers from inaccuracy and delays. Human visual and judgment limitations, coupled with complex scenarios during treatment, make precise evaluation of sterilization effects difficult. Real-time feedback is also unattainable, meaning issues may only be detected after it is too late to adjust treatment parameters, resulting in suboptimal or failed outcomes.

Therefore, achieving real-time monitoring of sterilization effects in non-incineration medical waste treatment to ensure safety and efficacy remains an urgent technical challenge.

### SUMMARY

To address the above technical problems-specifically, the inability to monitor sterilization effects in real time and the inefficiency and safety risks of manual monitoring in existing non-incineration medical waste treatment—the present application provides a method and system for monitoring sterilization effect of non-incineration medical waste treatment, aiming to enable real-time monitoring during the treatment process to ensure safety and timeliness.

In a first aspect, the present application provides a method for monitoring sterilization effect of non-incineration medical waste treatment, the method includes:
S1: acquiring a real-time temperature within a medical waste treatment chamber;
S2: acquiring a target sterilization logarithmic value;
S3: acquiring image data within the medical waste treatment chamber;
S4: determining whether a sterilization target can be achieved within a preset time based on analysis results of the real-time temperature, the target sterilization logarithmic value, and the image data;
S51: maintaining a current sterilization program if the sterilization target can be achieved; and
S52: adjusting parameters of the current sterilization program if the sterilization target cannot be achieved.

Preferably, step S4 includes:
S41: constructing a sterilization effect prediction model based on the real-time temperature and the target sterilization logarithmic value;
S42: determining whether a sterilization anomaly occurs within the medical waste treatment chamber based on the image data; and
S43: determining whether the sterilization target can be achieved within the preset time based on calculation results of the sterilization effect prediction model and the determination of whether the sterilization anomaly occurs.

Preferably, the sterilization effect prediction model is:

$$t = \frac{ln ( 10 ) \cdot {log}_{10} ( \frac{{N}_{0}}{{N}_{x}} )}{A exp ( - \frac{{E}_{0}}{R \cdot T} )}$$

where t is the calculated sterilization time, log₁₀

$$( \frac{{N}_{0}}{{N}_{x}} )$$

is the target sterilization logarithmic value, log₁₀

$$( \frac{{N}_{0}}{{N}_{X}} ) \geq 4 ,$$

N₀ is the initial microbial quantity, N_{X} is the target microbial quantity, A is the pre-exponential factor of the Arrhenius equation, Eₐ is the activation energy, R is the gas constant, and T is the real-time temperature within the medical waste treatment chamber.

Preferably, determining whether the sterilization target can be achieved within the preset time includes:
if the sterilization time is less than or equal to the preset time and no anomaly is detected in the medical waste treatment chamber, determining that the sterilization target can be achieved; and
if the sterilization time exceeds the preset time or an anomaly is detected in the medical waste treatment chamber, determining that the sterilization target cannot be achieved.

Preferably, step S2 includes:
S21: detecting the initial microbial quantity N₀ and the target microbial quantity N_{X} using a viability detection reagent; and
S22: calculating the target sterilization logarithmic value according to log₁₀

$$( \frac{{N}_{0}}{{N}_{x}} ) .$$

Preferably, the sterilization anomaly includes one or more of uneven spray water distribution, uneven stacking of medical waste, leakage of the medical waste treatment chamber, localized overheating in the medical waste treatment chamber, smoke in the medical waste treatment chamber, sparks in the medical waste treatment chamber, accumulation of spray water in the medical waste treatment chamber, or overheating of medical waste.

Preferably, adjusting parameters of the current sterilization program includes one or more of increasing the temperature within the medical waste treatment chamber, extending spray duration of spray water, or increasing a stirring speed of the medical waste.

Preferably, steps S1, S2, and S3 are performed simultaneously.

Preferably, the monitoring method further includes: repeating steps S1 to S4 after a set time following step S52.

### BRIEF DESCRIPTION OF THE DRAWINGS

The features and advantages of the present application will become clearer with reference to the accompanying drawings, which are schematic and not to be construed as limiting the application. In the drawings:

FIG. 1 is a flowchart of the method for monitoring sterilization effect of non-incineration medical waste treatment according to the present application; and

FIG. 2 is a structural diagram of the monitoring system for non-incineration medical waste treatment according to the present application.

### DETAILED DESCRIPTION OF THE EMBODIMENTS

To clarify the objectives, technical solutions, and advantages of the embodiments of the present application, the following describes the embodiments in detail with reference to the accompanying drawings. The described embodiments are illustrative and do not encompass all possible implementations. Other embodiments derived by those skilled in the art without creative effort shall fall within the scope of the present application.

Addressing the issues identified in the background-specifically, the lack of real-time sterilization monitoring and reliance on inefficient, unsafe manual methods in non-incineration medical waste treatment—the present application provides a method and system for real-time monitoring of sterilization effects to ensure safety and timeliness.

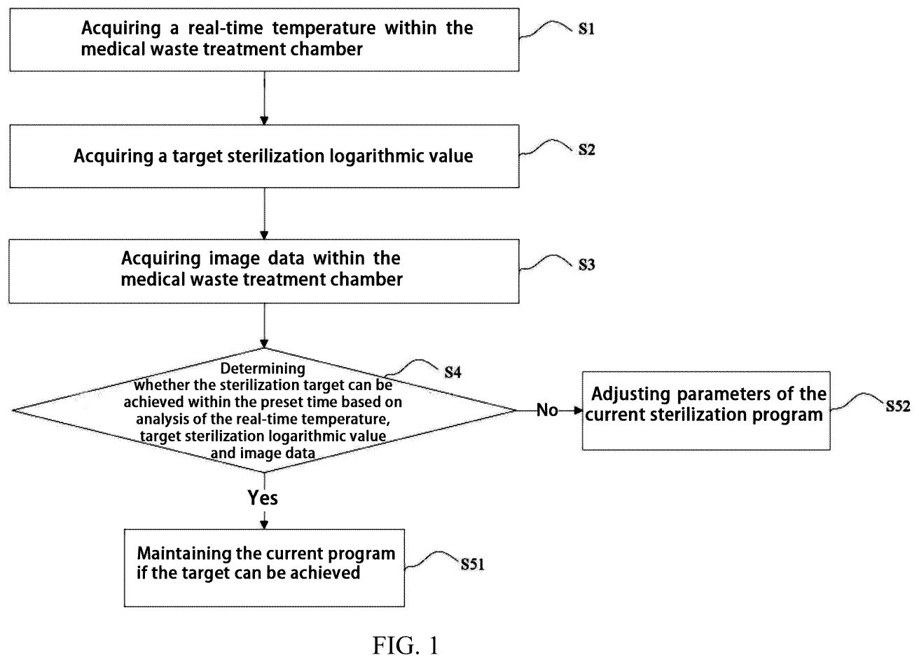

As shown in FIG. 1, the method for monitoring sterilization effect of non-incineration medical waste treatment includes:
S1: acquiring a real-time temperature within the medical waste treatment chamber.

Specifically, temperature sensors may be installed in the chamber to detect real-time temperatures. Multiple sensors may be placed at different positions, and their average temperature may be used as input for subsequent analysis.
S2: acquiring a target sterilization logarithmic value.

Preferably, viability detection reagents are used to detect microbial quantities. Step S2 includes: S21: detecting the initial microbial quantity N₀ and the target microbial quantity N_{X} (i.e., the post-sterilization microbial quantity) using viability detection reagents;
S22: Calculating the target sterilization logarithmic value using log₁₀

$$( \frac{{N}_{0}}{{N}_{x}} ) .$$

In one implementation, log₁₀

$$( \frac{{N}_{0}}{{N}_{x}} ) \geq 4 ,$$

indicating a minimum sterilization efficiency of 99.99% to meet regulatory requirements.
S3: acquiring image data within the medical waste treatment chamber.

Image collectors may be installed in the chamber to capture real-time image data for analysis. Multiple collectors may enhance coverage and accuracy.

Steps S1, S2, and S3 are executed simultaneously. Alternatively, they may be performed sequentially or in varying order, all of which fall within the scope of the application.

Data from sensors may be transmitted to a PLC (Programmable Logic Controller) for analysis and control.
S4: determining whether the sterilization target can be achieved within the preset time based on analysis of the real-time temperature, target sterilization logarithmic value, and image data.

Step S4 includes:
S41: constructing a sterilization effect prediction model based on the real-time temperature and target sterilization logarithmic value.

The model is:

$$t = \frac{ln ( 10 ) \cdot {log}_{10} ( \frac{{N}_{0}}{{N}_{x}} )}{A exp ( - \frac{{E}_{0}}{R \cdot T} )}$$

where ln(10) converts the logarithmic base from natural to base 10, A and Eₐ are determined via experimental data fitting, and exp

$$( - \frac{{E}_{a}}{R \cdot T} )$$

reflects temperature's impact on sterilization rate.

Higher temperatures reduce sterilization time.
S42: determining sterilization anomalies via image data analysis.

Anomalies include uneven spray distribution, waste stacking imbalance, chamber leakage, localized overheating, smoke, sparks, spray water accumulation, or waste overheating. Image collectors and PLC analysis identify these anomalies.
S43: judging achievability of the sterilization target within the preset time based on the model results and anomaly status.

Specifically:
if the calculated time≤preset time and no anomalies exist, the target can be achieved;
if the calculated time>preset time or anomalies exist, the target cannot be achieved.
S51: maintaining the current program if the target can be achieved.
S52: adjusting parameters (e.g., increase temperature, extend spray time, or enhance stirring speed) if the target cannot be achieved.

It should be noted that in the event of an abnormality within the medical waste treatment chamber, parameters of the current sterilization program can be adjusted first. Following step S52, after a set time, steps S1 to S4 are repeated, and a determination is subsequently made as to whether the abnormality persists. If the abnormality persists, processing of the medical waste may be suspended to allow manual investigation of the cause of the abnormality. The identified cause is then compared with data collected by the image collector and causes identified through PLC analysis to verify whether the root cause matches.

It should be noted that the method of the present application may be executed by a single device, such as a computer or a server. Alternatively, the method may be applied to a distributed scenario where multiple devices collaborate to complete the method. In such a distributed scenario, one device among the multiple devices may execute only one or more steps of the method, and the devices interact with each other to accomplish the method.

It should be noted that the above describes specific embodiments of the present application. Other embodiments fall within the scope of the appended claims. In some cases, actions or steps recited in the claims may be performed in an order different from that described in the embodiments and still achieve desired results. Additionally, processes depicted in the drawings do not necessarily require the specific or sequential order shown to achieve desired results. In some implementations, multitasking and parallel processing may also be feasible or advantageous.

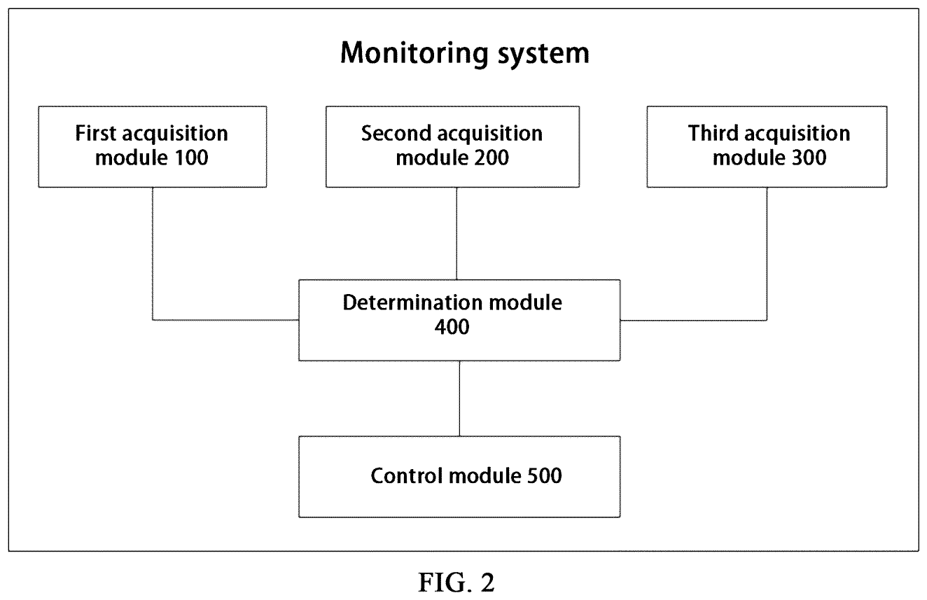

To achieve the same objective, corresponding to the method of any of the above embodiments, an embodiment of the present application further provides a monitoring system for sterilization effect of non-incineration medical waste treatment, as shown in FIG. 2. The monitoring system includes:
a first acquisition module 100 configured to acquire a real-time temperature within a medical waste treatment chamber;
a second acquisition module 200 configured to acquire a target sterilization logarithmic value;
a third acquisition module 300 configured to acquire image data within the medical waste treatment chamber;
a determination module 400 configured to determine whether a sterilization target can be achieved within a preset time based on analysis results of the real-time temperature, the target sterilization logarithmic value, and the image data; and
a control module 500 configured to maintain a current sterilization program when the sterilization target can be achieved within the preset time and adjust parameters of the current sterilization program when the sterilization target cannot be achieved within the preset time.

For clarity, the above apparatus is described by dividing it into functional modules. However, during implementation, the functions of these modules may be integrated into one or more software and/or hardware components. For example, the first acquisition module 100, second acquisition module 200, and third acquisition module 300 may be integrated into a single acquisition module to acquire the real-time temperature, target sterilization logarithmic value, and image data within the medical waste treatment chamber.

The apparatus in the above embodiments is used to implement the corresponding control method described in the preceding embodiments and shares the beneficial effects of the method embodiments, which will not be reiterated here.

For descriptive convenience, the above apparatus is divided into functional units. However, during implementation, the functions of these units may be integrated into one or more software and/or hardware components.

Those skilled in the art should understand that embodiments of the present application may be provided as methods, apparatuses, or systems. Thus, the application may take the form of a hardware-only embodiment, a software-only embodiment, or a combination of software and hardware.

It should also be noted that the terms “comprises,” “includes,” or any variation thereof are intended to cover non-exclusive inclusion, such that a process, method, product, or device comprising a list of elements includes not only those elements but also other elements not explicitly listed or inherent to such process, method, product, or device. Without further limitation, the phrase “includes a . . . ” does not exclude the presence of additional identical elements in the process, method, product, or device.

The embodiments of the present application are described in a progressive manner, with identical or similar parts cross-referenced between embodiments. Each embodiment focuses on differences from other embodiments. For system embodiments, descriptions are simplified as they largely correspond to method embodiments, and relevant details may be referenced from the method embodiments.

Those skilled in the art should understand that the discussion of any embodiment above is exemplary and not intended to imply that the scope of the disclosure (including the claims) is limited to these examples. Under the principles of the disclosure, technical features from different embodiments may be combined, steps may be performed in any order, and numerous other variations—as described in one or more aspects of the embodiments—exist but are omitted for brevity.

Although the present application has been described with reference to specific embodiments, many substitutions, modifications, and variations will be apparent to those skilled in the art based on the foregoing description.

One or more embodiments of the present application aim to cover all such substitutions, modifications, and variations falling within the broad scope of the appended claims. Therefore, any omissions, modifications, equivalent substitutions, or improvements made within the spirit and principles of the application shall fall within the protection scope of the present disclosure.

1. A monitoring method for monitoring sterilization effect of non-incineration medical waste treatment, comprising:
S1: acquiring a real-time temperature within a medical waste treatment chamber;
S2: acquiring a target sterilization logarithmic value;
S3: acquiring image data within the medical waste treatment chamber;
S4: determining whether a sterilization target is able to be achieved within a preset time based on analysis results of the real-time temperature, the target sterilization logarithmic value, and the image data;
wherein step S4 comprises:
S41: constructing a sterilization effect prediction model based on the real-time temperature and the target sterilization logarithmic value, the sterilization effect prediction model being:
$$t = \frac{ln ( 10 ) \cdot {log}_{10} ( \frac{{N}_{0}}{{N}_{x}} )}{A exp ( - \frac{{E}_{0}}{R \cdot T} )}$$
wherein t is a calculated sterilization time, log₁₀
$$( \frac{{N}_{0}}{{N}_{x}} )$$
is a target sterilization logarithmic value, log₁₀
$$( \frac{{N}_{0}}{{N}_{x}} ) \geq 4 ,$$
N₀ is an initial microbial quantity, N_{X} is a target microbial quantity, A is a pre-exponential factor of the Arrhenius equation, Eₐ is activation energy, R is a gas constant, and T is the real-time temperature within the medical waste treatment chamber;
S42: determining whether a sterilization anomaly occurs within the medical waste treatment chamber based on the image data;
S43: determining whether the sterilization target is able to be achieved within the preset time based on calculation results of the sterilization effect prediction model and the determination of whether the sterilization anomaly occurs; wherein determining whether the sterilization target is able to be achieved within the preset time comprises:
if the sterilization time is less than or equal to the preset time and no anomaly is detected in the medical waste treatment chamber, determining that the sterilization target is able to be achieved;
if the sterilization time exceeds the preset time or an anomaly is detected in the medical waste treatment chamber, determining that the sterilization target is not able to be achieved;
S51: maintaining a current sterilization program if the sterilization target is able to be achieved; and
S52: adjusting parameters of the current sterilization program if the sterilization target is not able to be achieved.

2. The monitoring method for monitoring sterilization effect of non-incineration medical waste treatment according to claim 1, wherein step S2 comprises:
S21: detecting the initial microbial quantity N₀ and the target microbial quantity N_{X} using a viability detection reagent; and
S22: calculating a specific value of the target sterilization logarithmic value according to log₁₀
$$( \frac{{N}_{0}}{{N}_{x}} ) .$$

3. The monitoring method for monitoring sterilization effect of non-incineration medical waste treatment according to claim 1, wherein the sterilization anomaly comprises one or more of uneven spray water distribution, uneven stacking of medical waste, leakage of the medical waste treatment chamber, localized overheating in the medical waste treatment chamber, smoke in the medical waste treatment chamber, sparks in the medical waste treatment chamber, accumulation of spray water in the medical waste treatment chamber, or overheating of medical waste.

4. The monitoring method for monitoring sterilization effect of non-incineration medical waste treatment according to claim 1, wherein adjusting parameters of the current sterilization program comprises one or more of increasing the temperature within the medical waste treatment chamber, extending spray duration of spray water, or increasing a stirring speed of the medical waste.

5. The monitoring method for monitoring sterilization effect of non-incineration medical waste treatment according to claim 1, wherein steps S1, S2, and S3 are performed simultaneously.

6. The monitoring method for monitoring sterilization effect of non-incineration medical waste treatment according to claim 1, further comprising: repeating steps S1 to S4 after a set time following step S52.

7. The monitoring method for monitoring sterilization effect of non-incineration medical waste treatment according to claim 2, further comprising: repeating steps S1 to S4 after a set time following step S52.

8. The monitoring method for monitoring sterilization effect of non-incineration medical waste treatment according to claim 3, further comprising: repeating steps S1 to S4 after a set time following step S52.

9. The monitoring method for monitoring sterilization effect of non-incineration medical waste treatment according to claim 4, further comprising: repeating steps S1 to S4 after a set time following step S52.

10. The monitoring method for monitoring sterilization effect of non-incineration medical waste treatment according to claim 5, further comprising: repeating steps S1 to S4 after a set time following step S52.

11. A monitoring system implementing the monitoring method for monitoring sterilization effect of non-incineration medical waste treatment according to claim 1, comprising:
a first acquisition module, configured to acquire a real-time temperature within a medical waste treatment chamber;
a second acquisition module, configured to acquire a target sterilization logarithmic value;
a third acquisition module, configured to acquire image data within the medical waste treatment chamber;
a determination module, configured to determine whether a sterilization target is able to be achieved within a preset time based on analysis results of the real-time temperature, the target sterilization logarithmic value, and the image data; and
a control module, configured to maintain a current sterilization program when the sterilization target is able to be achieved within the preset time and adjust parameters of the current sterilization program when the sterilization target is not able to be achieved within the preset time.

12. A monitoring system implementing the monitoring method for monitoring sterilization effect of non-incineration medical waste treatment according to claim 2, comprising:
a first acquisition module, configured to acquire a real-time temperature within a medical waste treatment chamber;
a second acquisition module, configured to acquire a target sterilization logarithmic value;
a third acquisition module, configured to acquire image data within the medical waste treatment chamber;
a determination module, configured to determine whether a sterilization target is able to be achieved within a preset time based on analysis results of the real-time temperature, the target sterilization logarithmic value, and the image data; and
a control module, configured to maintain a current sterilization program when the sterilization target is able to be achieved within the preset time and adjust parameters of the current sterilization program when the sterilization target is not able to be achieved within the preset time.

13. The monitoring system according to claim 11, wherein in the method implemented by the monitoring system, the sterilization anomaly comprises one or more of uneven spray water distribution, uneven stacking of medical waste, leakage of the medical waste treatment chamber, localized overheating in the medical waste treatment chamber, smoke in the medical waste treatment chamber, sparks in the medical waste treatment chamber, accumulation of spray water in the medical waste treatment chamber, or overheating of medical waste.

14. The monitoring system according to claim 11, wherein in the method implemented by the monitoring system, adjusting parameters of the current sterilization program comprises one or more of increasing the temperature within the medical waste treatment chamber, extending spray duration of spray water, or increasing a stirring speed of the medical waste.

15. The monitoring system according to claim 11, wherein in the method implemented by the monitoring system, steps S1, S2, and S3 are performed simultaneously.

16. The monitoring system according to claim 11, wherein the method implemented by the monitoring system further comprises: repeating steps S1 to S4 after a set time following step S52.

17. The monitoring system according to claim 12, wherein the method implemented by the monitoring system further comprises: repeating steps S1 to S4 after a set time following step S52.

18. The monitoring system according to claim 13, wherein the method implemented by the monitoring system further comprises: repeating steps S1 to S4 after a set time following step S52.

19. The monitoring system according to claim 14, wherein the method implemented by the monitoring system further comprises: repeating steps S1 to S4 after a set time following step S52.

20. The monitoring system according to claim 15, wherein the method implemented by the monitoring system further comprises: repeating steps S1 to S4 after a set time following step S52.
